(19) United States
(12) Patent Application Publication
(10) Pub. No.: US 2024/0226245 A9
(43) Pub. Date: Jul. 11, 2024

(54) ZIP-IN TECHNOLOGY FOR ANTIVIRAL THERAPEUTIC NANOFORMULATIONS

(71) Applicant: PAROLE LABORATORIES INC., Dorval (CA)

(72) Inventors: Oleg NEPOTCHATYKH, Dorval (CA); Evguenia NEPOTCHATYKH, Montreal (CA); Olga NEPOTCHATYKH, Dorval (CA)

(21) Appl. No.: 17/969,590

(22) Filed: Oct. 19, 2022

(51) Int. Cl. A61K 38/46; C12N 9/22; C12N 15/11; C12N 15/90

(52) U.S. Cl. CPC A61K 38/465; C12N 9/22; C12N 15/11; C12N 15/907; C12N 2310/20; C12N 2800/80

(57) ABSTRACT

Nanoparticles capable of delivering active pharmaceutical compounds (payload) into cells and methods of preparing same; wherein the payload is ionized and combined with a first polymer ion of an opposite charge resulting in the formation of an initial molecular assembly. The initial molecular assembly is then combined with a second polymer ion having an opposite charge and a different size than the first polymer ion, resulting in the formation of a secondary molecular assembly, wherein the payload becomes trapped between the first and the second polymer ions. The non-conjugated charged segments of the secondary molecular assembly are then repeatedly combined with additional polymer ions of alternating opposite charges to such segments charges and pre-determined size to extend and branch the molecular assembly resulting in the formation of a nanoparticle.

### ELECTRONIC FILE—SEQUENCE LISTING

This application hereby incorporates by reference in its entirety the material of the electronic Sequence Listing, in ASCII format. The ASCII copy is a 12 KB XML file created on May 4, 2023, entitled “107354-003_Sequence_Listing”.

### TECHNICAL FIELD

The present technology relates to a drug delivery system nanotechnology capable of delivering CRISPR (Clustered Regularly Interspaced Short Palindromic Repeat) based antiviral system into cells of humans and animals. CRISPR based antiviral system encapsulated into a biocompatible polysaccharide shell in the form of a liquid nanoformulation suitable for the pathogen's genome-targeted treatment of the most acute viral infections including COVID-19 in humans and animals.

### BACKGROUND

### Introduction

In human history, from time to time, deadly pandemics occurred, part of pandemics caused by bacterial infections and some—by viral infections. In the last century, many antibiotics were invented that allowed effective treatment of bacterial diseases. However, until the present time, the only effective protection against viral infections are vaccines. In case if a patient was not vaccinated and infected with a virus, then only symptomatic treatment is possible (ex. reduce fewer). Typically, the human immune system can provide a proper response to an unknown virus. However, this is a slow process, and the viral infection may cause complications and sometimes death. To date proposed many active compounds intended for the treatment of viral infections, but they have low efficiency, and they are useful only for limited viral types. The development of effective vaccines is a challenging and complicated task, and it requires a different approach for each viral infection and takes a long time to implement. In modern history, most pandemics as Spanish Flu, Asian Flu, AIDS, H1N1 Swine Flu, West African Ebola, Zika Virus and actual COVID-19 all caused by viral infections. There is no doubt that in several years a new deadly virus appears. The absence of vaccines and active therapeutic agents for newly appeared viruses makes them severely dangerous.

A broad research in the area of CRISPR-Cas based technology opens an excellent opportunity to design antiviral therapeutic agents. CRISPR technology is a powerful tool for gene therapy, gene editing, and antiviral protection and therapy. The CRISPR-Cas systems may be directed to target a specific viral RNA or DNA inside cells of a patient in case of acute viral infection, providing a fast and effective cure. CRISPR-Cas systems are present in prokaryotes as part of an adaptive immune system that permits prokaryotes to defend against an invasion of viruses. By incorporating a short sequence of a previously encountered phage or other virus sequences in their genome, prokaryotes use CRISPR-Cas systems to recognize and destroy an invader upon subsequent encounter [1]. These systems adapted by scientists and used to specifically target and cleave DNA or RNA sequences in many different organisms. This technology can now be applied in genome editing, gene therapy, viral detection and inhibition, RNA knockdown and others. CRISPR-Cas system consists of two components: a CRISPR-associated endonuclease (Cas) protein, which is capable of making a single-stranded or double-stranded DNA cleavage and a single guide RNA (sgRNA) which has the required information to associate with the Cas protein and to recognize the sequence to be cut. Many CRISPR-Cas systems exist, and each has its advantages and requirements in different applications. Mostly used Cas proteins in the CRISPR-Cas systems are Cas9, Cas12 and Cas13. CRISPR-Cas9 system can be used for manipulating genes of interest in gene therapy and targeting and inhibiting DNA viruses [2]. CRISPR-Cas12 system can be used to detect tiny amounts of DNA in a mixture [2]. The CRISPR-Cas13 system targets the only RNA and, therefore, can be used to target and inhibit RNA viruses. The fact that Cas13 is inactive against DNA makes CRISPR-Cas13 system save for use in patients and can be used for therapy to affect gene expression without changing the genome by targeting RNA messengers [2].

CRISPR-Cas system can be used in three forms. The first form is the Cas endonuclease protein and crRNA as a complex. The second is CasRNA, combined with crRNA. The CasRNA can express required Cas endonuclease. The third is a plasmid, designed in a way that it expresses both crRNA and Cas endonuclease inside human cells.

### Genetic Editing, Gene Therapy, DNA-Virus and Retrovirus Detection and Inhibition

For study purposes or therapeutic purposes, the editing of a genome is a type of genetic engineering in which DNA is inserted, deleted, modified or replaced. In gene therapy, the purpose is to correct a mutation. The manipulation of genomes relies on homologous recombination (HR), where a donor template DNA that has homologous sequences to the target DNA is used to replace a part of the target DNA and therefore introduce a change in the genome. While HR based gene manipulations by themselves are useful in some organisms, it is not very successful in human cell lines because of the low efficiency of homologous recombination in human cells [3]. With the combination of CRISPR-Cas technology and HR, specific genome editing in human cells can successfully be achieved. CRISPR-Cas9 system is a type II system where the Cas9 protein makes a blunt cut in double-stranded DNA, which then can be repaired by either non-homologous end joining or homologous recombination with a donor DNA template and this edits a specific part of DNA [2]. This system can be used for manipulating genes of interest and can be used in gene therapy. Genome editing using the CRISPR-Cas9 system has been successfully employed in a variety of model organisms, human embryonic and induced pluripotent stem cells, as well as in human adult stem cells [3]. For the induction of a double-stranded break by CRISPR-Cas9, to be specific, a sgRNA that guides the Cas9 protein to a specific location must be carefully designed. For the binding of the sgRNA to the genomic targets, a Protospacer Adjacent Motif (PAM) sequence located directly downstream of the target, on the opposite DNA strand, is necessary. The sgRNA must include the PAM sequence 5′NGG-3′ for Cas9, where N is any nucleotide [4].

Then, there is the CRISPR RNA (crRNA) segment of the sgRNA that is complementary to the genomic target, that binds to the desired segment of the DNA. This crRNA is about 18-20 nucleotides (nt) long. Then, there is the transactivating CRISPR RNA (tracrRNA) that permits for the sgRNA to set itself into the Cas9 protein and results in the crRNA-Cas9 interaction. For the guide RNA to exist in a single RNA and contain all the necessary segments, a linker loop segment is added to link the crRNA and the tracrRNA. When the use of the CRISPR-Cas9 system produces a double-stranded break in DNA, the cells try to repair it, and two types of repairs can occur, the Non-Homologous End Joining (NHEJ) and Homology-Directed Repair (HDR) [4]. NHEJ ligates the DNA ends back together, but this process is error-prone and, in most cases, results in an insertion or deletion of nucleotides (indels), if this region is part of a gene, the resulting protein is no longer functional. This process is known as gene knockout [4]. When replacing the targeted element with a different sequence (knock-in), a DNA donor template must be supplied to the cell. Following the double-stranded break, the cell will use this template to repair the DNA via homologous recombination, by incorporating a portion of the donor template where the break has occurred and thereby incorporating desired changes into the target region [4].

The CRISPR-Cas9 system can be used to target viral DNA if this system is present in the cell and has the appropriate crRNA segment of the sgRNA to target the virus. When CRISPR-Cas9 induces a double-stranded break in the viral DNA, a disruption of the essential virus protein-coding regions and/or cis-regulatory elements in a virus genome occurs this results in blockage of virus genome replication, which leads to the establishment of the antiviral defence inside a host cell [5]. The off-target activity of the CRISPR-Cas9 system must be carefully evaluated by examining the potential host DNA target sequences with high sequence homology to the viral target sequence. This system can be used to target and inhibit DNA viruses and retroviruses. To inhibit a DNA virus engineering of sgRNA is required, a sgRNA is programmed to recognize the DNA of the virus and thus can inactivate the virus before its genome replication.

The CRISPR-Cas9 system can also be used to target and to inhibit a retrovirus after the reverse transcription of the viral RNA has occurred.

### RNA-Based Virus Inhibition

CRISPR-Cas system can be used to target RNA instead of DNA. CRISPR-Cas13 system is a type VI system which once bound to the single-stranded RNA, unleashes a nonspecific RNase activity and destroys all nearby RNA regardless of their sequence [2].

Similar to the CRISPR-Cas9 sgRNA, the guide RNA for the CRISPR-Cas13 is composed of around 64-66 nucleotides crRNA sequence, which includes 24 to 30 nucleotide spacer region which is complementary to the target region [6]. Cas13 contains two higher eukaryotes and prokaryotes nucleotide-binding domains (HEPN), which together form the ribonuclease-active site, allowing it to work as an RNA-guided, RNA-targeting CRISPR system [6]. In bacteria, similar to a PAM sequence, the Cas13 protein requires a Protospacer Flanking Sequence (PFS), and the protein cleaves the targeted RNA by the two HEPN domains downstream of the PFS sequence [6]. However, in mammalian cells, no PFS restrictions have been observed for the Cas13 orthologs, including Cas13a, Cas13b, and Cas13d [7]. For this project, we propose the use of the Cas13b ortholog since a higher knockdown 90-95% of luciferase reporter has been published [8].

The CRISPR-Cas13b system could potentially be used to target and inhibit RNA-based viruses such as COVID-19.

If CRISPR-Cas13 system present inside the cell and if it contains the crRNA sequence complementary to the single-stranded viral RNA sequence such as COVID-19 or influenza, this system will bind to the viral RNA and with the two HEPN domains cleave the viral RNA and therefore inactivate it.

COVID-19 virus, also known as SARS-CoV-2, is a part of a large Coronavirus (CoVs) family of viruses [9]. Genomic characterization of this COVID-19 strain of coronavirus indicated an 82% nucleotide match with the human SARS virus, and this is why this strain is also known as SARS-CoV-2 [9]. CoVs are the cause of the respiratory, hepatic, nervous system, and gastrointestinal system diseases in humans [9]. Coronaviruses have a non-segmented, positive-sense RNA genome of about 30 kb in length [10]. Their genome has a 5′ cap structure and a 3′ poly (A) tail, allowing it to act as an mRNA for the translation of their replicase polyproteins [10]. The gene for the replicase polyproteins, the non-structural proteins (nsp), occupies two-thirds of the genome, about 20 kb [10]. On the other hand, there are structural and accessory proteins, which make up only about the rest 10 kb of the sequence [10]. The coronavirus genome is organized in the following way, 5′-leader-UTR-relicase-S(spike)-E(Envelope)-M(Membrane)-N(Nucleocapsid)-3′UTR-poly(A) tail [10]. The replicase gene encodes rep1a and rep1b, which express two polyproteins, pp1a and pp1ab [10]. The pp1a and pp1ab contain the nsp 1-11 and 1-16, respectively [10]. These polyproteins after translation are cleaved into individual nsps. Many of these nsps assemble into Replicase-Transcriptase Complex (RTC) to create an environment suitable for RNA synthesis and are responsible for RNA replication and transcription of the sub-genomic RNAs [10]. The genome of COVID-19 has been sequenced and is listed by the National Center for Biotechnology Information (NCBI)[12]. The replicase polyproteins sequences for the COVID-19 virus are known (ORF1ab). In addition, the individual nsp sequences of the COVID-19 viral genome have been identified. However, to date, there is no anti-COVID-19 CRISPR-Cas-associated complex engineered, which is the subject of the present technology.

There are several advantages of using the CRISPR-Cas13 system. When CRISPR-Cas13 binds to a targeted RNA besides cleaving the targeted RNA, it unleashes a non-specific RNase activity and destroys all nearby RNA regardless of their sequence [2]. However, this non-specific RNase activity is not displayed in eukaryotic cells, such as human cells [6]. Such activity could be especially beneficial in cases of massive viral invasion. Another advantage is that the Cas13 protein can only be active on a single-stranded RNA since it lacks helicase activity necessary for opening up double-stranded RNA regions for guided binding [7]. One of the significant advantages of Cas13, if it is used in human cells as a therapeutic defence against RNA viruses such as COVID-19, is that CRISPR-Cas13 does not possess genome editing properties; it does not bind to or cleave DNA. Therefore, there is no risk of off-target effects that could be irreversible.

An essential part of anti-COVID-19 CRISPR-Cas13b crRNA associated complex, is crRNA, and it must be engineered to be complementary to a region (a target point) in the COVID-19 RNA. CRISPR-Cas13b associated complex will cleave the COVID-19 RNA at the chosen target point. It has been previously reported that many positive-stranded RNA viruses to which belong COVID-19 contain mechanism of repairing their 3′ends; these mechanisms are similar to telomerase activity [11]. Therefore, randomly chosen points of cleavage on the COVID-19 RNA may not result in inhibition of the virus, because in many cases, a virus may repair itself.

### CRISPR-Associated Complex In-Vivo Delivery

A big problem exists for therapeutic antiviral use of CRISPR based technology because CRISPR-Cas-associated complex cannot be directly administered into a patient. This complex can be successfully administered into a unicellular organism, but to deliver it into every cell of a multicellular organism, including a human is the problem. Cas endonuclease protein and crRNA as any other RNAs break down quickly in a patient's bloodstream. Several attempts were made to deliver CRISPR-Cas-associated complex using transfection, microinjection, electroporation and viral technologies, useful in-vitro but without much success in patients. Many side effects observed, including the death of patients. Besides, this makes the technology extremely complicated [13]. The CRISPR-Cas based technology is missing a key element; it requires a simple and effective delivery vehicle of CRISPR-Cas-associated complex inside the patient's cells. Inventors are not aware of successful delivery vehicles suitable for delivery of CRISPR-Cas-associated complex inside the patient's cells. The present technology is devoted to this problem. In this technology proposed technology of nanoencapsulation of CRISPR-Cas-associated complex into polysaccharide particles. Polysaccharides are a large class of biomolecules, and they have been studied for a long time as prospective drug delivery vehicles due to their excellent biocompatibility and metabolism. Rapid and efficient cellular uptake of nanoparticles based on many classes of polysaccharides has been reported [14]. There are many publications devoted to the encapsulation of different drugs into polysaccharide particles. Most of them pointed to cationic polysaccharides chitosan and its derivative trimethyl chitosan (TMC) because they can be cross-linked to form ionic gel particles for example with tripolyphosphate (TPP). The TMC-TPP particles formed because of “ionic gelation” where TPP anion cross-links two TMC molecules, turning the solution into a gel if the concentration of TMC is high or into particle suspension at a low TMC concentration [15-17]. Such particles are well characterized and regarded as a prospective drug encapsulation material. Many attempts were made to encapsulate different drugs into TMC-TPP nanoparticles, for example CSKSSDYQC peptide [18], vitamins (B9, B12 and C) [19], proteins [20], anti-neuroexcitation peptide [21], ovalbumin antigen [22], Tetanus Toxoid [23], Insulin [24], lactosyl-norcantharidin [25], Vancomycin [26] and Paclitaxel [27]. Typically, a researcher adds a drug substance into a TMC solution and then mixes it with a TPP solution, formed particles studied immediately or freeze-dried. Despite the attractive simplicity of the synthesis of loaded particles, there are many difficulties in this process. The most important is that TMC-TPP particles possess extremely low stability. Reported a strong influence of electrolytes on drug release kinetics from loaded TMC particles [15, 24]. TMC, in combination with TPP, forms particles in deionized water; however, those particles dissolve upon the addition of an electrolyte, as Phosphate Buffer Saline (PBS), required for IV formulation. IV formulation must have pH 7.4 and salinity of 285 milli-osmoles per kilogram. At these conditions, TMC-TPP particles dissolve entirely, which makes the preparation of IV formulation impossible. If formulation prepared without a salt (PBS), the TMC-TPP particles will disintegrate as soon as they contact with blood during injection because blood has the same ionic strength as PBS. Also reported by many and observed by us, that TMC-TPP particles are not stable even in pure water and due to hydrolysis of TPP disintegrate during several days, making TMC-TPP particles useless for pharmaceutical formulations. Another problem is the ionic gelation itself, also called cross-linking. In this process, polysaccharide molecules become linked in random places, and this creates a continuous jelly or gel particles. The problem is that such a structure is very permeable even for large molecules. The gel particle is unable to protect nor to retain encapsulated in it drug molecules. Even very large molecules such as proteins, RNAs and DNAs are able to move freely through such gels and this effect used in gel electrophoresis. There were hundreds of attempts to find a “magic” polysaccharide or a “magic combination” that will allow proper drug retention, its protection and delivery, but there is no such thing found, because all researchers use the same “gelation” technique. The solution is not in a material, but in the technology.

To date, inventors are not aware of a drug encapsulation technology that provides controlled molecular assembly of a drug—polysaccharide complex into a stable multilayer nanoparticle with the drug payload permanently trapped in the centre of the particle providing excellent cellular uptake and adequate protection of the payload molecule from premature release and degradation caused by RNAse and other biomolecule attacks.

### SUMMARY

From a broad aspect, the present technology relates to the synthesis of a drug delivery vehicle in the form of a “giant” macromolecular complex of a nanoparticle size. In certain embodiments, the nanoencapsulation of the present technology includes controlled assembly of CRISPR-Cas—polysaccharide nano-complexes in the way that the CRISPR-Cas system is zipped in between two ionic polysaccharide molecules with opposite charges and further coated with compact polysaccharide layers providing adequate protection of CRISPR-Cas system from RNAse and ferment degradation in a patient's bloodstream. The encapsulated CRISPR-Cas system may include: a) Cas endonuclease (as Cas9 or Cas13) with crRNA targeted to a specific virus (for example COVID-19); b) Cas RNA and crRNA, or their conjugate; c) plasmid designed to express both Cas endonuclease and anti-COVID-19 crRNA accordingly.

From another aspect, the present technology relates to an anti-viral, in particular anti-COVID-19 therapeutic agent consisting of CRISPR-Cas-associated complex nanoencapsulated in a polysaccharide shell with a non-specific body cellular uptake in the form of a stable liquid suspension suitable for IV or other administration in human and/or in animals.

From another aspect, the present technology relates to twelve guide RNA (crRNA) sequences targeted to the COVID-19 virus essential for anti-COVID-19 CRISPR complex.

From another aspect, the present technology relates to a method of nanoencapsulation of a payload comprising: ionizing of a payload; combining the ionized payload with a polymer ion of an opposite charge resulting in formation of initial molecular assembly; combining the initial molecular assembly with a second polymer ion of an opposite charge to the first polymer ion in the way that the payload trapped between the two polymer ions attached each to the other by means of opposite charges resulting in formation of a molecular assembly.

In certain embodiments, the ionization of the payload is controlled by the pH of a reaction media.

In other embodiments, the two said polymer ions are of different physical sizes resulting in the formation of charged segments in said molecular assembly.

In certain embodiments, an increase in the molecular assembly size is provided by the attachment to the charged segment of the molecular assembly of a next polymer ion of an opposite charge to the said charged segment. In certain embodiments, said polymer ion is larger than the charged segment in the molecular assembly providing the formation of same or increased number of charged segments of reversed polarity within said molecular assembly.

In certain embodiments, addition of a polymer ion with an opposite to said charged segments polarity is repeated a number of times, as polyanion, then polycation and then polyanion again and so on, providing further increase in size of said molecular assembly which results in formation of a spherical nanoparticle due to folding of it conjugated chain.

In certain embodiments, the physical sizes of polymer ions used for molecular assembly size amplification are of the same size or a size ratio multiple of two or three.

In certain embodiments, the charged segments in said molecular assembly or spherical nanoparticle brought into interaction with polymer ions of smaller than said charged segments size, preferably of a broad size distribution, providing end-capping of said molecular assembly, i.e., eliminating of non-conjugated charged segments as the last step in the synthesis of the loaded nanoparticle.

In other embodiments, the pH and salinity of the final product are modified to match properties, required for a pharmaceutical formulation.

In other embodiments, then two polymeric ions are any ionic polymers in a charged state, such as any polymers with repeated ionizable acidic and basic groups able to form polyanions and polycations accordingly, linear or branched, either synthetic, semisynthetic or natural.

In certain embodiments, the two polymeric ions are ionic carbohydrates, including but not limited to chitosan, trimethyl chitosan, alginate, heparin, hyaluronate, glucuronan, fucans, fucoidans, carrageenans, galactans, agarans, ulvans and ionic derivatives of other polysaccharides.

In certain embodiments, one or both of polymer ions are chemically combined with biomarker molecule or molecules, such as proteins, antibodies, peptides and so on, providing selective cellular uptake of the encapsulated payload and the use of said product as a vehicle for targeted drug delivery including but not limited to targeted cancer therapy.

In certain embodiments, said end-capping short polymer ions are chemically combined with biomarker molecule or molecules, such as antibodies, peptides and so on, providing the presence of said biomarkers on the surface of said molecular assembly or a nanoparticle and resulting in selective cellular uptake of the encapsulated payload and the use of said product as a vehicle for targeted drug delivery including but not limited to targeted cancer therapy.

In certain embodiments, the particle surface charge and its polarity are controlled by a size and a nature of said end-capping short polymer ions.

In certain embodiments, the payload is a small molecule with acidic or basic groups, ionizable molecule, a peptide, a protein, an RNA, a DNA, plasmid or their combination. In other embodiments, the payload is any CRISPR-Cas-associated complex or a part of it. In further embodiments, the payload is CRISPR-Cas-associated complex with COVID-19 or any other virus signature and its use for antiviral therapy in humans and animals. In certain implementations of these embodiments, the CRISPR-Cas-associated complex consists essentially of Cas13 endonuclease of all forms and crRNA complementary to the ORF1ab region in COVID-19 virus genome that encodes the replicase polyproteins, and its use for anti-COVID-19 therapy. In further implementations of these embodiments said crRNA is targeted to nsp protein sequences nsp12, nsp14A2 and nsp16 of the COVID-19 genomic sequence and is further used for anti-COVID-19 therapy.

In other embodiments, the CRISPR-associated complex consists of any Cas protein+crRNA, or CasRNA+crRNA or a plasmid, designed to express both Cas protein and crRNA in body cells and is further used for anti-COVID-19 or other antiviral therapy.

In other embodiments, in the process and the product of the present technology, the payload is a CRISPR-associated complex designed for human or animal gene therapy or gene editing and its use accordingly.

In other embodiments, the payload is a solid nanoparticle including but not limited to bismuth sulphide and bismuth telluride. In certain embodiments, the product is for use as an X-ray contrast agent.

From another aspect, the present technology relates to a method of nanoencapsulation of an active pharmaceutical compound (payload) comprising: ionizing a payload; combining the ionized payload with a first polymer ion of an opposite charge resulting in the formation of an initial molecular assembly; combining the initial molecular assembly with a second polymer ion of an opposite charge to the first polymer ion such that the payload is trapped between the first and the second polymer ions attached each to the other by means of opposite charges, resulting in formation of a molecular assembly.

In certain embodiments, ionization of the payload is controlled by the pH of a reaction media.

In other embodiments, the first and the second polymer ions are of different physical sizes resulting in the formation of charged segments in said molecular assembly.

In certain embodiments, the method further comprises increasing a size of the molecular assembly by attaching to the charged segment of the molecular assembly a next polymer ion having an opposite charge to said charged segment. In certain implementations of these embodiments, said next polymer ion is larger than the charged segment in the molecular assembly and provides the formation of a same or an increased number of charged segments of reversed polarity within said molecular assembly.

In other embodiments, the method further comprises adding a polymer ion with an opposite charge to said charged segments polarity and repeating said adding a number of times with a polyanion, then a polycation and then a polyanion again and so on, to provide a further increase in size of said molecular assembly, which results in formation of a spherical nanoparticle due to folding of its conjugated chain.

In certain embodiments, the physical sizes of polymer ions used for molecular assembly size amplification are of the same size or a size ratio multiple of two or three.

In other embodiments, the method further comprises interacting the charged segments in said molecular assembly or spherical nanoparticle with end-capping short polymer ions of smaller than said charged segments size, to provide end-capping of said molecular assembly. In certain implementations of these embodiments, the end-capping short polymer ions are of a broad size distribution.

In other embodiments, the method further comprises matching the pH and salinity of the molecular assembly or spherical nanoparticle to properties required for a pharmaceutical formulation.

In other embodiments, the first and second polymer ions are polymers with repeated ionizable acidic and basic groups able to form polyanions and polycations. In further embodiments, the first and second polymer ions may be linear or branched. In yet further embodiments, the first and second polymer ions may be synthetic, semisynthetic or natural. In certain implementations of these embodiments, the first and second polymer ions are ionic carbohydrates. In certain embodiments, the ionic carbohydrate is a chitosan, a trimethyl chitosan, an alginate, a heparin, a hyaluronate, a glucuronan, a fucan, a fucoidan, a carrageenan, a galactan, an agaran, or an ulvan, or ionic derivatives thereof.

In certain embodiments, any one or more of the first or the second polymer ions are chemically combined with at least one biomarker molecule to provide selective cellular uptake of the encapsulated payload. In other embodiments, said end-capping short polymer ions are chemically combined with at least one biomarker molecule, to provide the presence of said biomarkers on the surface of said molecular assembly or spherical nanoparticle and result in selective cellular uptake of the encapsulated payload. In certain implementations of these embodiments, the at least one biomarker is a protein, an antibody, or a peptide.

In other embodiments, a surface charge and a polarity of the molecular assembly or spherical nanoparticle are controlled by a size and nature of said end-capping short polymer ions.

In certain embodiments, the payload is a small molecule with acidic or basic groups, an ionizable molecule, a peptide, a protein, a RNA, a DNA, a plasmid or any combinations thereof. In other embodiments, the payload is a CRISPR-Cas-associated complex. In further embodiments, the payload is a CRISPR-Cas-associated complex with COVID-19 or any other virus signature. In certain implementations of these embodiments, the CRISPR-Cas-associated complex consists essentially of Cas13 endonuclease of all forms and crRNA complementary to the ORF1ab region in the COVID-19 virus genome that encodes the replicase polyproteins. In certain embodiments, said crRNA is targeted to nsp protein sequences nsp12, nsp14A2 and nsp16 of the COVID-19 genomic sequence.

In other embodiments, the CRISPR-Cas-associated complex consists of any Cas protein+crRNA, CasRNA+crRNA, or a plasmid designed to express both a Cas protein and a crRNA in human or animal cells.

In further embodiments, the payload is a CRISPR-associated complex designed for human or animal gene therapy or gene editing.

In yet further embodiments, the payload is an active pharmaceutical compound.

### BRIEF DESCRIPTION OF THE DRAWINGS

Reference will now be made to the accompanying drawings.

FIG. 1 illustrates the net charge vs pH of vancomycin.

FIG. 2 illustrates the net charge vs pH of Cas9 endonuclease.

FIG. 3 illustrates the net charge vs pH of Cas13 endonuclease

FIG. 4 illustrates the encapsulation steps 2a to 4a: payload molecule ionization and reaction with polyanion.

FIG. 5 illustrates encapsulation step 5a: initial molecular assembly reacted with polycation.

FIG. 6 illustrates encapsulation steps 6a to 8a: extension and branching of molecular assembly.

FIG. 7 illustrates encapsulation step 9a: end-capping of non-conjugated areas with short ionic oligomers.

FIG. 8 illustrates encapsulation step 9a: end-capping of non-conjugated areas with short ionic oligomers that have cell targeting biomarkers attached.

### DETAILED DESCRIPTION

The present technology relates to a delivery vehicle for anti-COVID-19 CRISPR-Cas-associated complex and other drugs (payloads) in the form of a multilayered nanoparticle, in which payload molecule zipped in between the two oppositely charged polysaccharide macromolecules and then wrapped by several layers of oppositely charged polysaccharides, so the payload is located in the centre of the formed nanoparticle, and is isolated from external attacks by RNAse and ferments. The said nanoparticle can be made with biomarkers on its surface for targeted delivery.

The proposed drug delivery vehicle engineered as follows. First, payload molecule ionized by appropriate pH of the reaction media and it assumes approximately a linear shape due to the electrostatic repulsion, if the payload molecule is large and possesses multiple charges. Next, the payload combined with a polymer ion (I) which also fully ionized, unwound and of an opposite to the payload polarity at the same pH. During this stage due to the electrostatic forces between molecules the payload molecule orients in a parallel to the polymer ion and forms an ionic complex with the polymer ion (I). Next, a second (II) polymer ion added to the reaction. It is also in fully ionized state and its polarity opposite to the polarity of the polymer ion (I). The polymer ion (II) orients in parallel and finally forms ionic complex with the polymer ion (I) with attached to it payload. As a result, the payload molecule becomes effectively zipped in between the two ionic polymer chains, and it loses its mobility without any chemical modification, which is essential for this method and the final product. The use of two polymer ions (I) and (II) of specific sizes and at precise stoichiometric ratio results in the molecular assembly with charged, non-conjugated segments on larger polymer ion chain. Following addition of a complimentary by charge polymer ions results in further extension of the molecular assembly. The size of a complimentary polymer ion added the last into the reaction mixture must provide the formation of molecular assembly with two new charged, non-conjugated segments to allow extension of the molecular assembly. The lastly added polymer ion could be the same as (I), or (II), or a different ionic polymer by nature. Then next complimentary polymer ion (with the opposite charge to the previous) added and the molecular assembly becomes larger accordingly. Those steps can be repeated multiple times. The two conjugated each with the other ionic polymers form a double-chained assembly interconnected by ionic bonds and as a result, the polymeric ions neutralize their net charges. The absence of charged groups on the double polymer chain allows it to curl into a ball, thus forming a spherical nanoparticle with the payload inside. The surface of the nanoparticle still contains multiple non-conjugated segments of the lastly added ionic polymer and carry positive or negative charge accordingly. If a surface charge is not desirable, then end-capping performed by the addition of short ionic polymers. One or several short ionic molecules conjugate with long charged segments of the last ionic polymer and form fully closed neutral double-chained assemblies. End-capping of the nanoparticle with a short ionic polymer that includes chemically attached protein or antibody as a biomarker is an excellent way to engineer cell or organ targeted drug delivery vehicles. Choice of ionic polymers required for this technology and other essential requirements and considerations provided within example explanation section below.

### Anti-COVID-19 CRISPR-Cas-Associated Complex:

To ensure that the cleavage of COVID-19 RNA produced by CRISPR-Cas13b crRNA associated complex is not repairable and that this cleavage will lead to the total viral inhibition, we propose to construct crRNA in the CRISPR-Cas13b system to be complementary to the region of the viral genome that encodes the replicase polyproteins, the ORF1ab region. By targeting the regions in the ORF1ab, we are destroying the mechanism by which the virus is able to replicate itself and transcribe its proteins, which will lead to complete inhibition of the virus. Specifically, we propose to target the nsp protein sequences that are important in the viral RNA replication. We propose to target nsp12 of COVID-19 genomic sequence, which is located from 13442-16236nt in COVID-19 genome. Nsp12 encodes RNA-dependent RNA polymerase. Also, we propose to target the sequence for nsp14A2 with the location 18039-19620nt, which encodes the 3′ to 5′ exonuclease, involved in replication fidelity and N7-methyltransferase activity [10]. In addition, we propose to target the sequence nsp16 at the 20659-21553nt, this sequence encodes the 2′O-methyltransferase protein [12].

In the present technology we disclosed twelve guide RNA sequences (crRNA) engineered for the CRISPR-Cas13b targeting the required nsp protein sequences in COVID-19 genome. Using the CRISPR-Cas13b system, we also propose to target the whole coronavirus family of viruses by creating the crRNAs complementary to the conserved sequences of the family.

### EXAMPLES

The following are examples of encapsulation of drugs and CRISPR-Cas-associated complexes or their components as positively and negatively charged payloads with explanations and requirements required for the successful use of the proposed technology.

### Steps of Nanoencapsulation Technology:

Step 1—determination of the payload molecule suitability.

Step 2—determination of the pH range, at which the payload molecule carries its maximal net charge and charge polarity.

Next steps depend on the charge polarity of the payload molecule.

### Encapsulation of a Positively Charged Payload:

Step 3a—preparation of payload, polyanion and polycation solutions at required concentrations and the pH, determined in the step 2.

Step 4a—combining calculated volume of polyanion with reaction media followed with slow addition of payload solution.

Step 5a—addition of calculated volume of polycation—zipping in payload.

Step 6a—secondary addition of calculated volume of polyanion solution, creating branching and further extension of molecular assembly.

Step 7a—secondary addition of calculated volume of polycation solution, creating branching and further extension of molecular assembly.

Step 8a—repeating steps 6a and 7a until desired size of particles obtained.

Step 9a—end-capping.

Step 10a—addition of components of Phosphate Buffer Saline (PBS) into the reaction mixture at required quantities to pH 7.4 and osmolality to 285 milli-osmoles per kilogram.

### Encapsulation of a Negatively Charged Payload:

Step 3b—preparation of payload, polyanion and polycation solutions at required concentrations and the pH, determined in the step 2.

Step 4b—combining calculated volume of polycation with reaction media followed with slow addition of payload solution.

Step 5b—addition of calculated volume of polyanion—zipping in payload.

Step 6b—secondary addition of calculated volume of polycation solution, creating branching and further extension of molecular assembly.

Step 7b—secondary addition of calculated volume of polyanion solution, creating branching and further extension of molecular assembly.

Step 8b—repeating steps 6a and 7a until desired size of particles obtained.

Step 9b—end-capping.

Step 10b—addition of components of Phosphate Buffer Saline (PBS) into the reaction mixture at required quantities to pH 7.4 and osmolality to 285 milli-osmoles per kilogram.

### Comments, Essential Requirements and Explanations for Steps of Disclosed Technology:

Step 1: Payload molecule must be ionizable, preferably be relatively large, and preferably hold multiple charges in an ionized state. Most drugs, peptides, proteins, RNAs, DNAs are ionizable and met those requirements. The present technology is not suitable for non-ionizable molecules. Solid materials of inorganic or organic nature can have charged surface and be suitable for encapsulation by proposed technology, and examples are silica, bismuth sulphide and bismuth telluride. On the surface of some other solid materials, charged groups can be created intentionally, as by oxidation, acylation and other ways. Said materials or particles of any size can be coated with polyanion-polycation layers using the same disclosed technology. The products, as solid nanoparticles coated with polysaccharide shell with attached antibodies, may have a practical interest in X-ray imaging, for example.

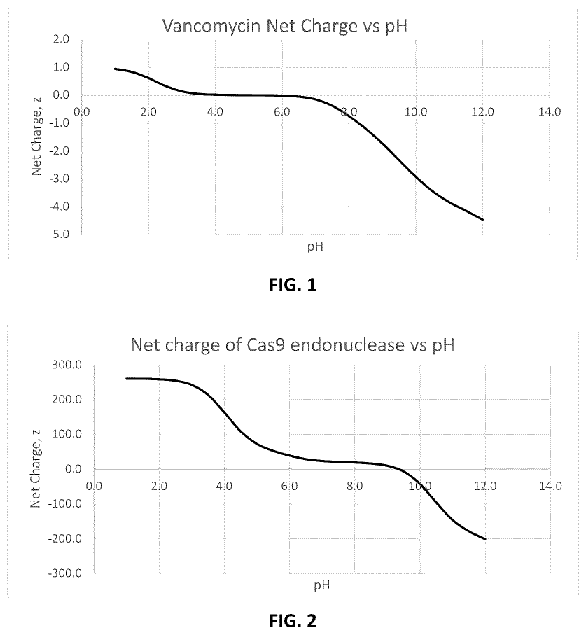

Step 2: This is an essential step. For short payload molecules with a single ionizable group is enough to know pKa, so the useful pH range is pH>pKa+1 for acidic compounds and pH<pKa−1 for basic compounds. For payloads that are macromolecules with multiple different ionizable groups, such as peptides and proteins, must be measured or calculated net charge vs pH dependence for the full pH range (0-14). Provided three examples, refer to FIGS. 1 to 3.

For the vancomycin example, there are two ranges of pH where it is ionized (FIG. 1), one is pH 1 and below and the second range is pH 8 and up. Vancomycin at pH ˜1 acquires net charge +1, however at pH 12, the net charge is −4.5. Therefore, vancomycin must be encapsulated at pH 12 by the disclosed method as a “negatively charged payload”. Note, in the range of pH from 3 to 7.5 it is impossible to encapsulate vancomycin because it has no charge.

The next example is Cas9 endonuclease protein. As per FIG. 2, this protein can be encapsulated at pH below 4 and above 11. An excessively high pH could cause protein denaturation, and so the pH 3-4 is the most favorable for encapsulation of Cas9 protein.

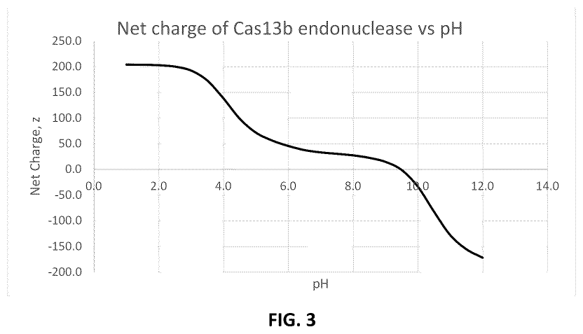

The third example is endonuclease Cas13, proposed in this technology as a part of CRISPR antiviral agent against COVID-19 and other RNA viruses, has approximately the same pH ranges for encapsulation as Cas9 (FIG. 3); however, it has a larger positive charge at a neutral pH making a final formulation more stable. Both Cas9 and Cas13 endonucleases have to be encapsulated at pH<4 as “positively charged payloads”.

Essential part of CRISPR antiviral agent proposed in this technology is encapsulation of RNA and DNA based payloads (crRNA, gRNA, CasRNA, plasmid . . . ). All RNAs and DNAs are acids by the nature with average pKa between 2.5 to 7.0. This means that at a pH above pKa+1 RNA molecule ionized almost completely. We propose encapsulation of RNAs and DNAs of all forms at a pH above pKa+1 as a “negatively charged payload”. Although Cas protein and crRNA, which are required for CRISPR-associated complex, could be encapsulated in the same process, however, the most stable product is obtained if Cas protein and crRNA encapsulated in two separate processes, as positively and negatively charged payloads accordingly. The products of those two processes (nanoparticle suspensions) are mixed in a final formulation.

Steps 3a-4a: For the technology, disclosed in the present application a correct choice of ionic polymers (polyanion and polycation) is essential. The particular choice of an ionic polymer pair used in this technology depends on manufacturing conditions as pH, required biocompatibility, stability, polymer metabolism and cell/organ targeting. In the process, disclosed in the present technology can be used all types of ionic polymers, synthetic, semi-synthetic and natural.

Reaction media is ultrapure water RNAse free with the addition of minimal amounts of pH modifiers, as H₃PO₄, CO₂, NaHCO₃, Na₂CO₃, and so on. pH must be adjusted in the way to create minimal possible ionic force in the media. The same applies to the preparation of payload and ionic polymer solutions. The polyanion used in step 4a must be in a fully ionized state. In the example of Cas13 encapsulation, the best pH determined at step 2 is pH 3 and below, so the polyanion to be ionized must have pKa<3-1. There is a broad choice of synthetic, natural and semisynthetic polymers that carry acidic groups. Natural polysaccharides with carboxylic groups such as alginic acid and glucuronan typically have pKa=3-3.5, so they can be used for this technology at a pH>4-4.5. For encapsulation of this particular protein, Cas13 required a polyanion with lower pKa, such as sulphated polysaccharides. There is an excellent choice of natural polysaccharides containing sulphate esters extracted from seaweeds such as fucans, fucoidans, carrageenans, galactans, agarans, and ulvans. Besides, neutral polysaccharides can be easily sulphated with a high yield of a product with required properties. Typical pKa of sulphated polysaccharides is about −2, and this means they all are strong acids and ionized at any pH.

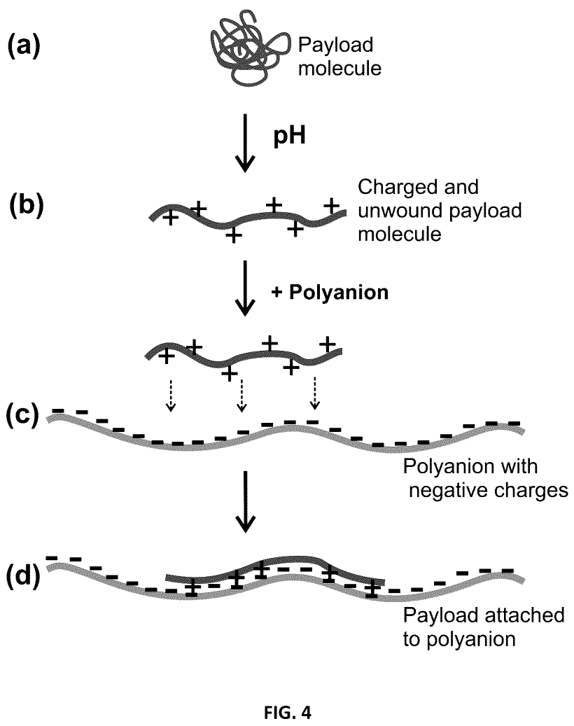

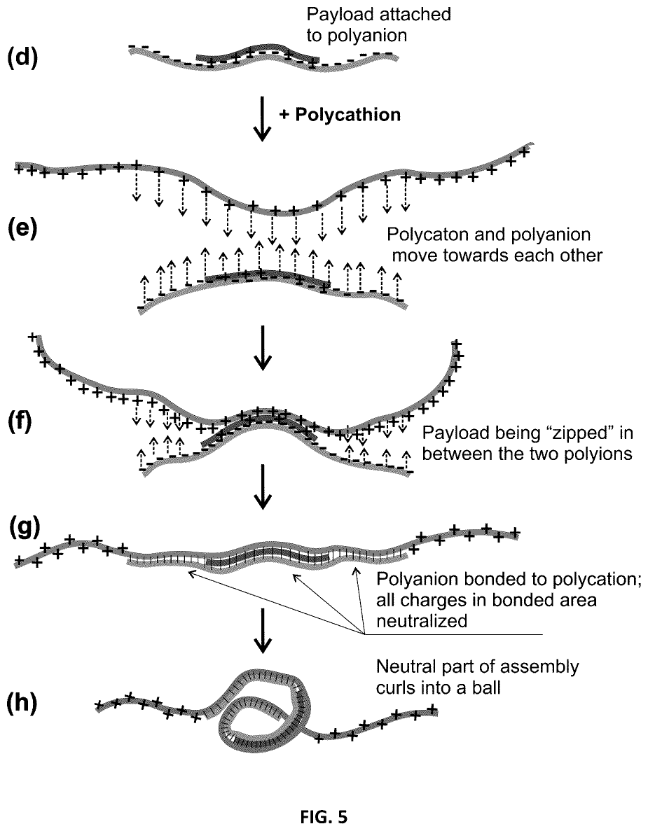

The size of charged and unwound polyanion, used for step 4a, must be larger, preferably in two times or more, than the size of charged payload molecule. This process is illustrated by the FIG. 4.

Step 5a: In this step, the polycation also must be in a fully ionized state, so its linear shape allows proper orientation and contact with the polyanion in parallel. Next, the polycation is attracted to polyanion, ionized groups of polyanion form bonds with ionized groups of polycation effectively trapping or “zipping” the payload molecule between them. The process is carried out at the same pH as per step 2, which is pH 3, in this example.

The choice of polycation for this step also depends on manufacturing conditions (pH), required biocompatibility, stability, polymer metabolism and cell/organ targeting. In this particular example can be used either chitosan or trimethyl chitosan. Both are fully ionized at pH 3. For this method is essential that the correct size ratio of used polyanion and polycation and the stoichiometry. As mentioned above, polyanion used in step 4a should preferably have size twice or more of the size of the payload molecule. For step 5a is essential, that the polycation used in this step has a size two times larger than polyanion in step 4a. This creates molecular assembly with two non-conjugated areas at both ends, required for molecular assembly extension. In case of ionic polymers with variable size, the size distribution curves of two ionic polymers must not overlap, otherwise fractionation of those polymers required. The geometrical size of a macromolecule is not the same as molecular weight, number of nucleotides, number of amino acids or degree of polymerization. The disclosed method requires that sizes of all used macromolecules are known, in particular of payload and especially of both ionic polymers. Size of all macromolecules used in this technology must be measured at the same pH as determined in step 2, i.e. at charged, unwound state using size exclusion chromatography, or gel-filtration chromatography.

Stoichiometry of step 5a: Amount of polycation in step 5a must be equimolar to polyanion in step 4a. The step 5a illustrated by FIG. 5.

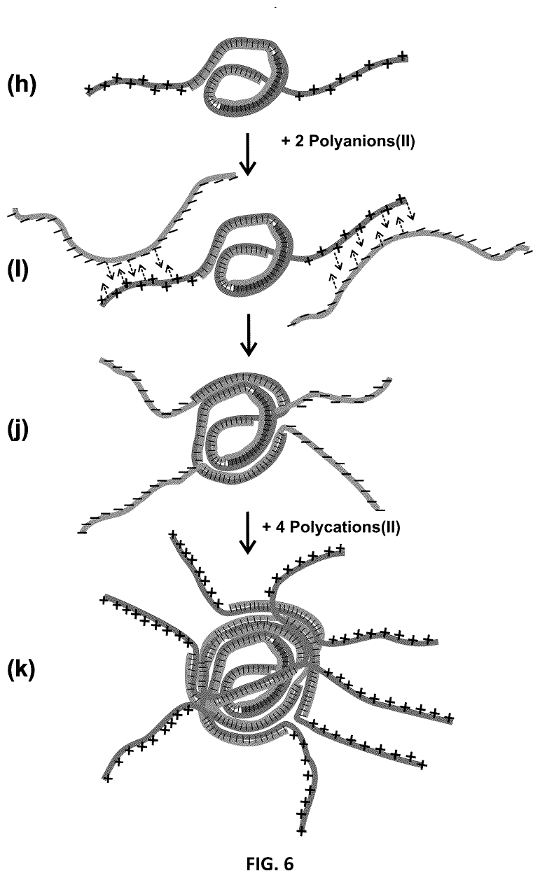

Steps 6a-8a: For extension of the molecular assembly obtained in step 5a can be used solutions of polyanion and polycation of the size larger than the size of non-conjugated areas of said molecular assembly. Extension of molecular assembly in steps 6a-8a with polymeric ions (polyanion and polycation) of sizes three times larger than non-conjugated areas doubles the number of new non-conjugated areas and requires doubling of amount of added polymer ion, i.e. molar amount of polyanion added in the step 6a must be two times of the amount of polycation used in the step 5a, and step 7a requires two times larger amount of polycation than the amount of polyanion in the step 6a and so on. Described conditions provide branching and extension of the molecular assembly as shown in FIG. 6.

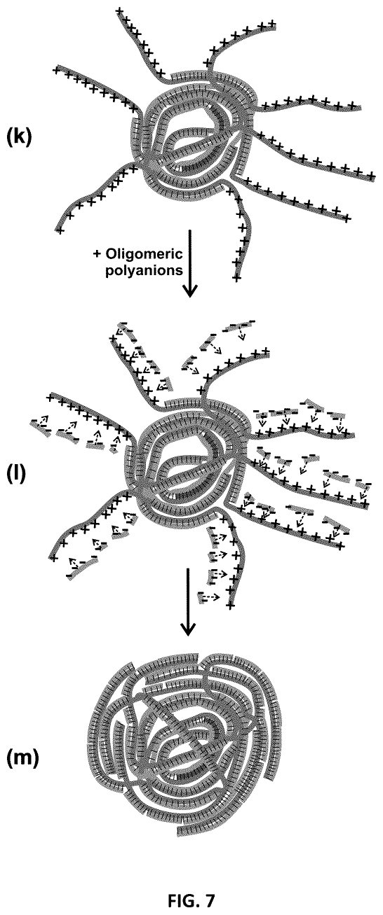

Step 9a: Short ionic polymers (oligomers) of the same nature as those used in previous steps, or different ionic polymers are suitable for end-capping. The polarity of end-capping oligomers must be opposite to the polarity of the last ionic polymer used in step 8a (FIG. 7).

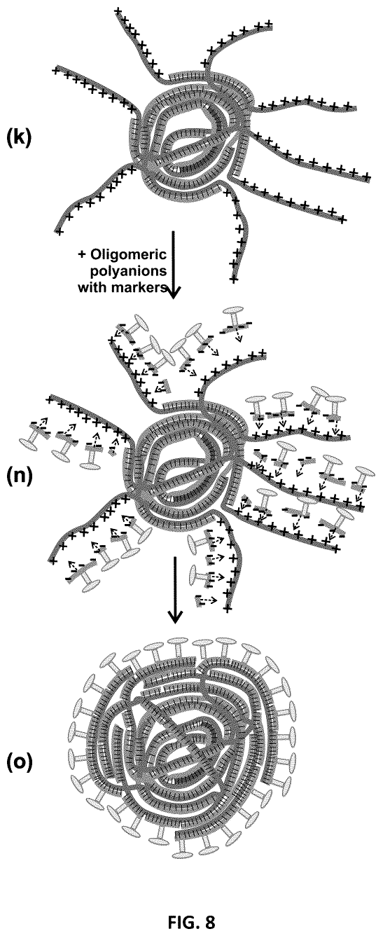

It is very advantageous to modify those oligomers with organ or cell targeting biomarkers. An example is end-capping ionic oligomer with chemically attached cancer cell monoclonal antibody, like trastuzumab, pertuzumab bevacizumab. With paclitaxel as a payload, product formulation can be used for targeted therapy of breast cancer (FIG. 8). Sizes of end-capping oligomer molecules can be different, preferably with a broad size distribution. In case of polysaccharides can be used a partial hydrolysate of the same ionic polymer as used in step 4a or 5a.

Step 10a: This step is adjustment of pH and salinity to the physiological levels, such as pH 7.4 and 285 milli-osmoles per kilogram. If required, the formulation can be concentrated by centrifugation and protective colloid can be added to stabilize a final formulation; therefore, similar sterile formulations manufactured by inventors are stable at +5° C. without protective colloid for a minimum of four years.

Encapsulation of a Negatively Charged Payload:

Step 3b: Encapsulation of a negatively charged payload molecules, such as RNA, DNA or plasmid includes the same steps as for positively charged payload, but with reversed order of use polycations and polyanions. Same requirements apply. Information below is an example of encapsulation of RNA as a negatively charged payload. All required solutions must be prepared in ultrapure RNAse-free water, pH of all solutions adjusted to the value, determined in step 2, in this example it is pH 8. RNA is extremely sensitive to the presence of ferments, known as RNAses. They are very common contaminants, present on human skin and everything touched by a bare hand is contaminated with following RNA destruction. This poses special requirements for raw materials, equipment, glassware and procedure to be RNAse-free.

Step 4b: Same requirements as for step 4a. In this particular example, reaction media has pH 8; the polycation must be fully ionized at this pH, so pKa>pH. Chitosan as polycation will not work because it has pKa ˜6.5. However, trimethyl chitosan with its quaternary ammonium groups is a strong base and ionized at pH 8.

Step 5b: Same requirements as for step 5a. As a polyanion in this example can be used almost any acidic polymer, this includes sulfated polysaccharides and carboxylic polysaccharides, such as alginic acid, because all such polymeric acids are ionized at pH 8.

Steps 6b-10b: Essentially the same as steps 6a-10a, but with reversed order of polyanions and polycations.

### Other Considerations:

Preferable that both polyanion and polycation have similar spatial distribution of charged groups along polymer chain.

In the disclosed technology can be used linear and branched ionic polymers. Although stoichiometry can become more complicated, branched ionic polymers especially suitable for large 3D payload molecules, such as plasmids.

### Anti-Covid-19 Intravenous Injectable Formulation Manufacturing Example:

Cas13b and crRNA nanoencapsulated in separate processes and then combined to form a final product.

### Positively Charged Payload Protocol

Prepare PBS buffer 5× concentrate, filter 0.22 μm pH 7.4, autoclave.

Dissolve 3.9 mg Na-A-carrageenan Mw 230,000 in 90 mL UHP water, adjust pH to 3, bring volume to 100 mL, filter 0.22 μm sterile.

Dissolve 15.2 mg Na-alginate hydrolysate (end-capper) average Mw 2500 in UHP water, pH 7 bring volume to 100 mL, filter 0.22 μm sterile.

Dissolve 24.7 μg Cas13b protein (dry weight) in 100 mL UHP water; and add ˜1 mL 0.5% H₃PO₄; to final pH 3, filter with sterile 0.22 μm filter system.

Dissolve 6.9 mg of 3-n-methylchitosan (TMC) Mw 400,000 in 90 mL UHP water, adjust pH to 3, bring volume to 100 mL, filter 0.22 μm sterile.

Dissolve 4.5 mg Na-alginate Mw 260000 in UHP water, pH 7 bring volume to 100 mL, filter 0.22 μm sterile.

Place 300 mL UHP sterile water in 500 mL sterile reactor, add 0.2 M H₃PO₄ to pH 3, add 1 mL carrageenan solution (2) and @ 3 ml/min add 100 mL Cas13 solution (4) while stirring @ 700 rpm, incubate for 1 hour at 35° C.

While stirring @ 700 rpm add 1 mL TMC (5) solution, incubate 1 hour at 35° C.

Adjust pH to 6.2 with 0.1 M Na₂CO₃

While stirring @ 700 rpm add 2 mL Na-alginate (6) solution, incubate 1 hour at 35° C.

While stirring @ 700 rpm add 4 mL TMC (5) solution, incubate 1 hour at 35° C.

While stirring @ 700 rpm add 8 mL Na-alginate (6) solution, incubate 1 hour at 35° C.

While stirring @ 700 rpm add 16 mL TMC (5) solution, incubate 1 hour at 35° C.

While stirring @ 700 rpm add 15 mL Na-alginate hydrolysate solution (3), incubate 1 hour at 35° C.

Add PBS concentrate 1:5 to the reaction mixture.

Adjust pH to 7.4 by sterile 0.2 M Na₂CO₃, to measure pH take 1 mL with sterile pipet, measure and discard.

### Negatively Charged Payload Protocol

For the following reaction prepare solution of 3.6 μg crRNA (dry weight) in 100 mL UHP RNAse-free water; add 0.5 mL 0.5 M NaHCO₃ to pH 8.

Dissolve 3.1 mg of TMC Mw=180,000 in 90 mL UHP RNAse-free water, add 1 mL 0.5 M NaHCO₃ to pH 8, bring volume to 100 mL, autoclave.

Check alginate (6) and TMC (5) and (18) solutions for RNAse activity, if passed-continue, if not—autoclave @135° C. for 30 min, re-check. Diethylpyrocarbonate not allowed as RNase decontamination.

Place 300 mL UHP water in 500 mL reactor, add 0.5 M NaHCO₃ to pH 8, add 1 mL TMC (18) solution and @ 3 mL/min add 100 mL crRNA solution while stirring @ 700 rpm, incubate for 1 hour at 35° C.

While stirring @ 700 rpm add 1 mL alginate (6) solution, incubate 1 hour at 35° C.

While stirring @ 700 rpm add 2 mL TMC (5) solution, incubate 1 hour at 35° C.

While stirring @ 700 rpm add 4 mL alginate (6) solution, incubate 1 hour at 35° C.

While stirring @ 700 rpm add 8 mL TMC (5) solution, incubate 1 hour at 35° C.

While stirring @ 700 rpm add 16 mL alginate (6) solution, incubate 1 hour at 35° C.

While stirring @ 700 rpm add 32 mL TMC (5) solution, incubate 1 hour at 35° C.

While stirring @ 700 rpm add 15 mL Na-alginate hydrolysate solution (3), incubate 1 hour at 35° C.

Add PBS concentrate 1:5 to the reaction mixture.

Product of (28) combine with product of (16) 1:1 to form the final formulation.

Encapsulation of CAS-13bRNA+crRNA system as well as CAS-13b−crRNA plasmid performed by negatively charged payload protocol only. Technologies of preparation and purification of Cas13b endonuclease, crRNA and plasmid are well-known art and are not a subject of the present technology, except anti-COVID-19 crRNA sequences disclosed herein. However, custom crRNA synthesis and its amplification is relatively cheap and straightforward; Cas13b protein can be cheaply expressed in E. coli bioreactors in industrial quantities, (same as it has done for insulin, for example); plasmid-manufacturing biotechnology is over 20 years and is not expensive nor complicated either.

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

All references cited in this specification, and their references, are incorporated by reference herein in their entirety where appropriate for teachings of additional or alternative details, features, and/or technical background.

### REFERENCES

A. A. Price, A. Grakoui, and D. S. Weiss, “Harnessing the Prokaryotic Adaptive Immune System as a Eukaryotic Antiviral Defense,” Trends Microbiol., vol. 24, no. 4, pp. 294-306, 2016, doi: 10.1016/j.tim.2016.01.005.

“CRISPR Systems: What's the Difference? CRISPR-Cas9; CRISPR-Cas12; CRISPR-Cas13,” https://www.cell.com/pb-assets/products/research-arc/infographics/CrisprVizInfo_vol1a.pdf

T. B. Meissner, P. K. Mandal, L. M. R. Ferreira, D. J. Rossi, and C. A. Cowan, “Genome editing for human gene therapy”, 1st ed., vol. 546, no. C. Elsevier Inc., 2014.

SYNTHEGO, eBook “CRISPR 101: your guide to understand CRISPR”, p. 45, 2011, https://www.synthego.com/resources

C. Lee, “CRISPR/Cas9-based antiviral strategy: Current status and the potential challenge,” Molecules, vol. 24, no. 7, 2019, doi: 10.3390/molecules24071349.

J. T. Granados-Riveron and G. Aquino-Jarquin, “CRISPR-Cas13 precision transcriptome engineering in cancer,” Cancer Res., vol. 78, no. 15, pp. 4107-4113, 2018, doi: 10.1158/0008-5472.CAN-18-0785.

“Cas13 @ Zenlab.Bio.”, https://zlab.bio/cas13.

D. B. T. Cox et al., “RNA editing with CRISPR-Cas13,” vol. 358, no. 6366, pp. 1019-1027, 2018, doi: 10.1126/science.aaq0180.RNA.

S. A. Hassan, F. N. Sheikh, S. Jamal, J. K. Ezeh, and A. Akhtar, “Coronavirus (COVID-19): A Review of Clinical Features, Diagnosis, and Treatment,” Cureus, vol. 12, no. 3, 2020, doi: 10.7759/cureus.7355.

H. J. Maier, E. Bickerton, and P. Britton, “Coronaviruses: Methods and protocols,” Coronaviruses Methods Protoc., vol. 1282, no. 1, pp. 1-282, 2015, doi: 10.1007/978-1-4939-2438-7.

P. D. Nagy, C. D. Carpenter, and A. E. Simon, “A novel 3′-end repair mechanism in an RNA virus,” Proc. Natl. Acad. Sci. U.S.A., vol. 94, no. 4, pp. 1113-1118, 1997, doi: 10.1073/pnas.94.4.1113.

SARS-CoV-2 (Severe acute respiratory syndrome coronavirus 2) Sequences, https://www.ncbi.nlm.nih.gov/genbank/sars-cov-2-seqs/

C. A. Lino, J. C. Harper, J. P. Carney, J. A. Timlin, “Delivering CRISPR: a review of the challenges and approaches,” Drug Deliv. 2018 November; 25(1):1234-1257.

S. Salatin, A. Y. Khosroushahi, “Overviews on the cellular uptake mechanism of polysaccharide colloidal nanoparticles,” J. Cell. Mol. Med. Vol 21, No 9, 2017 pp. 1668-1686)

K. Nagpal, S. K. Singh, D. N. Mishra, “Chitosan Nanoparticles: A Promising System in Novel Drug Delivery.”, Chem. Pharm. Bull. 58(11) 1423-1430 (2010)

Q. Gan, T. Wang, “Chitosan nanoparticle as protein delivery carrier-Systematic examination of fabrication conditions for efficient loading and release, Colloids and Surfaces B: Biointerfaces,” Volume 59, Issue 1, 2007, Pages 24-34, ISSN 0927-7765

M. Amidi, E. Mastrobattista, W. Jiskoot, W. E. Hennink, “Chitosan-based delivery systems for protein therapeutics and antigens.”, Adv Drug Deliv Rev. 2010 Jan. 31; 62(1):59-82.

G. Chen, D. Svirskis, W. Lu, M. Ying, Y. Huang, J. Wen, “N-trimethyl chitosan nanoparticles and CSKSSDYQC peptide: N-trimethyl chitosan conjugates enhance the oral bioavailability of gemcitabine to treat breast cancer,” Journal of Controlled Release, Volume 277, 2018, Pages 142-153

D. de Britde Britto, M. R. de Moura, F. A. Aouada, L. H. C. Mattoso, O. B. G. Assis, “N,N,N-trimethyl chitosan nanoparticles as a vitamin carrier system, Food Hydrocolloids,” Volume 27, Issue 2, 2012, Pages 487-493

F. Chen, Z. Zhang, Y. Huang, “Evaluation and modification of N-trimethyl chitosan chloride nanoparticles as protein carriers,” International Journal of Pharmaceutics, Volume 336, Issue 1, 2007, Pages 166-173

S. Wang, T. Jiang, M. Ma, Y. Hu, J. Zhang, “Preparation and evaluation of anti-neuroexcitation peptide (ANEP) loaded N-trimethyl chitosan chloride nanoparticles for brain-targeting.” Int J Pharm. 2010 Feb. 15; 386(1-2):249-55

S. Boontha, H. E. Junginger, N. Waranuch, A. Polnok and T. Pitaksuteepong, “Formation of particles prepared using chitosan and their trimethyl chitosan derivatives for oral vaccine delivery: Effect of molecular weight and degree of quaternization.”, Songklanakarin J. Sci. Technol. 32 (4), 363-371, July-August 2010

M. Zarifpour, F. Hadizadeh, M. Iman, M. Tafaghodi, “Preparation and Characterization of Trimethyl Chitosan Nanospheres Encapsulated with Tetanus Toxoid for Nasal Immunization Studies.”, Pharm Sci. 2013; 18(4):193-198

A. Jintapattanakit, T. Kissel and V. Buraphacheep Junyaprasert, “Effect of Tripolyphosphate on Physical and Enzymatic Stabilities of Insulin Loaded Nanoparticles of N-Trimethyl Chitosan.” Pharm Sci Asia; 35(1)

M. Guan, Y. Zhou, Q. L. Zhu, Y. Liu, Y. Y. Bei, X. N. Zhang, Q. Zhang, “N-trimethyl chitosan nanoparticle-encapsulated lactosyl-norcantharidin for liver cancer therapy with high targeting efficacy.”, Nanomedicine. 2012 October; 8(7):1172-81

J. Xu, B. Xu, D. Shou, X. Xia and Y. Hu, “Preparation and Evaluation of Vancomycin-Loaded N-trimethyl Chitosan Nanoparticles.”, Polymers 2015, 7(9), 1850-1870

R.-F. Song, X. Li, X. L. Cheng, A. R. Fu, Y. Wang, Y. J. Feng, Y. Xiong, “Paclitaxel-loaded trimethyl chitosan-based polymeric nanoparticle for the effective treatment of gastroenteric tumors.”, Oncology reports 32: 1481-1488, 2014

The contents of the above-cited references are incorporated herein by reference in their entirety.

1. A method of nanoencapsulation of a payload comprising:
a. ionizing a payload;
b. combining the ionized payload with a first polymer ion of an opposite charge resulting in the formation of an initial molecular assembly;
c. combining the initial molecular assembly with a second polymer ion of an opposite charge to the first polymer ion such that the payload is trapped between the first and the second polymer ions attached each to the other by means of opposite charges, resulting in formation of a molecular assembly.

2. The method of claim 1, wherein ionization of the payload is controlled by the pH of a reaction media.

3. The method of claim 1, wherein the first and the second polymer ions are of different physical sizes resulting in the formation of charged segments in said molecular assembly.

4. The method of claim 3, further comprising increasing a size of the molecular assembly by attaching to the charged segment of the molecular assembly a next polymer ion having an opposite charge to said charged segment.

5. The method of claim 4, wherein said next polymer ion is larger than the charged segment in the molecular assembly and provides the formation of a same or an increased number of charged segments of reversed polarity within said molecular assembly.

6. The method of claim 5, further comprising adding a polymer ion with an opposite charge to said charged segments polarity and repeating said adding a number of times with a polyanion, then a polycation and then a polyanion again and so on, to provide a further increase in size of said molecular assembly, which results in formation of a spherical nanoparticle due to folding of it conjugated chain.

7. The method of claim 5, wherein the physical sizes of polymer ions used for molecular assembly size amplification are of the same size or a size ratio multiple of two or three.

8. The method of claim 3, further comprising interacting the charged segments in said molecular assembly or spherical nanoparticle with end-capping short polymer ions of smaller than said charged segments size, to provide end-capping of said molecular assembly.

9. The method of claim 8, wherein the end-capping short polymer ions are of a broad size distribution.

10. The method of claim 1, further comprising matching the pH and salinity of the molecular assembly or spherical nanoparticle to properties required for a pharmaceutical formulation.

11. The method of claim 1, wherein the first and second polymer ions are polymers with repeated ionizable acidic and basic groups able to form polyanions and polycations.

12. The method of claim 11, wherein the first and second polymer ions are linear or branched.

13. The method of claim 11, wherein the first and second polymer ions are synthetic, semisynthetic or natural.

14. The method of claim 1, wherein the first and second polymer ions are ionic carbohydrates.

15. The method of claim 14, wherein the ionic carbohydrate is a chitosan, a trimethyl chitosan, an alginate, a heparin, a hyaluronate, a glucuronan, a fucan, a fucoidan, a carrageenan, a galactan, an agaran, or an ulvan, or ionic derivatives thereof.

16. The method of claim 1, wherein any one or more of the first or the second polymer ions are chemically combined with at least one biomarker molecule to provide selective cellular uptake of the encapsulated payload.

17. The method of claim 8, wherein said end-capping short polymer ions are chemically combined with at least one biomarker molecule, to provide the presence of said biomarkers on the surface of said molecular assembly or spherical nanoparticle and result in selective cellular uptake of the encapsulated payload.

18. The method of claim 16, wherein the at least one biomarker is a protein, an antibody, or a peptide.

19. The method of claim 8, wherein a surface charge and a polarity of the molecular assembly or spherical nanoparticle are controlled by a size and nature of said end-capping short polymer ions.

20. The method of claim 1, wherein the payload is a small molecule with acidic or basic groups, an ionizable molecule, a peptide, a protein, a RNA, a DNA, a plasmid, or an active pharmaceutical compound, or any combinations thereof.

21. The method of claim 1, wherein the payload is a CRISPR-Cas-associated complex.

22. The method of claim 1, wherein the payload is a CRISPR-Cas-associated complex with COVID-19 or any other virus signature.

23. The method of claim 22, wherein the CRISPR-Cas-associated complex consists essentially of Cas13 endonuclease of all forms and crRNA complementary to the ORF1ab region in the COVID-19 virus genome that encodes the replicase polyproteins.

24. The method of claim 23, wherein said crRNA is targeted to nsp protein sequences nsp12, nsp14A2 and nsp16 of the COVID-19 genomic sequence.

25. The method of claim 21, wherein the CRISPR-Cas-associated complex consists of any Cas protein+crRNA, CasRNA+crRNA, or a plasmid designed to express both a Cas protein and a crRNA in human or animal cells.

26. The method of claim 1, wherein the payload is a CRISPR-associated complex designed for human or animal gene therapy or gene editing.


SEQUENCE LISTING (12 sequences)

SEQ ID NO: 1 - RNA, 66 nt, synthetic construct
  source 1..66: mol_type = other RNA
tgtcaaaagccctgtatacgacatcagtacgttgtggaaggtccagttttgggggctattacaaca

SEQ ID NO: 2 - RNA, 66 nt, synthetic construct
  source 1..66: mol_type = other RNA
ttgaatttagtgtcaacactgaggtgtgtagttgtggaaggtccagttttgggggctattacaaca

SEQ ID NO: 3 - RNA, 66 nt, synthetic construct
  source 1..66: mol_type = other RNA
actatcaccataattttgaaggtcacagttgttgtggaaggtccagttttgggggctattacaaca

SEQ ID NO: 4 - RNA, 66 nt, synthetic construct
  source 1..66: mol_type = other RNA
atttgcttgttccaattactacagtagctcgttgtggaaggtccagttttgggggctattacaaca

SEQ ID NO: 5 - RNA, 66 nt, synthetic construct
  source 1..66: mol_type = other RNA
agcacactcattagctaatctatagaaacggttgtggaaggtccagttttgggggctattacaaca

SEQ ID NO: 6 - RNA, 66 nt, synthetic construct
  source 1..66: mol_type = other RNA
caacccataaggtgagggttttctacatcagttgtggaaggtccagttttgggggctattacaaca

SEQ ID NO: 7 - RNA, 66 nt, synthetic construct
  source 1..66: mol_type = other RNA
aacaattatgtgttgaacctttctacaagagttgtggaaggtccagttttgggggctattacaaca

SEQ ID NO: 8 - RNA, 66 nt, synthetic construct
  source 1..66: mol_type = other RNA
gcaattccaaaataggcatacaccatctgtgttgtggaaggtccagttttgggggctattacaaca

SEQ ID NO: 9 - RNA, 66 nt, synthetic construct
  source 1..66: mol_type = other RNA
atgatgtctacagacagcaccacctaaattgttgtggaaggtccagttttgggggctattacaaca

SEQ ID NO: 10 - RNA, 66 nt, synthetic construct
  source 1..66: mol_type = other RNA
cgaagtgtcccatgagcttataaagatcaggttgtggaaggtccagttttgggggctattacaaca

SEQ ID NO: 11 - RNA, 66 nt, synthetic construct
  source 1..66: mol_type = other RNA
tgcttcagatgatgacgcattcacattagtgttgtggaaggtccagttttgggggctattacaaca

SEQ ID NO: 12 - RNA, 66 nt, synthetic construct
  source 1..66: mol_type = other RNA
aacagctgtacctggtgcaactcctttatcgttgtggaaggtccagttttgggggctattacaaca